(19) United States
(12) Patent Application Publication
(10) Pub. No.: US 2010/0056881 A1
(43) Pub. Date: Mar. 4, 2010

(54) Method and Apparatus For Acute Cardiac Monitoring

(75) Inventors: Imad Libbus, St. Paul, MN (US); Yatheendhar D. Manicka, Woodbury, MN (US); Scott T. Mazar, Woodbury, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/546,918

(22) Filed: Aug. 25, 2009

Related U.S. Application Data
(60) Provisional application No. 61/093,088, filed on Aug. 29, 2008.

(51) Int. Cl. A61B 5/0408; A61B 5/00; A61B 5/0402; A61B 5/053; A61B 5/085; A61B 5/0468

(52) U.S. Cl. 600/301; 600/300; 600/509; 600/547; 600/529; 600/515; 600/382

(57) ABSTRACT

A device for monitoring a patient from the back of the patient comprises a support configured to adhere to the back of the patient, at least two electrodes supported with the support, circuitry coupled to the at least two electrodes to measure a signal from the at least two electrodes, and circuitry to transmit the signal wirelessly. The support and the at least two electrodes may be placed on at least one of a lower back or between shoulder blades of the patient, which can help to reduce pressure on the patient when the device is worn for an extended period, for example 1 week. Placement of the adherent device in at least one of these locations can improve patient comfort, for example by decreasing pressure to the skin of the patient from the device when the patient lies supine. The device may also provide lumbar support, for example when placed on the lumbar of the patient.

### CROSS-REFERENCES TO RELATED APPLICATIONS

The present application claims the benefit under 35 USC 119(e) of U.S. Provisional Application No. 61/093,088 filed Aug. 29, 2008; the full disclosure of which is incorporated herein by reference in its entirety.

### STATEMENT AS TO RIGHTS TO INVENTIONS MADE UNDER FEDERALLY SPONSORED RESEARCH AND DEVELOPMENT

Not Applicable

### BACKGROUND OF THE INVENTION

1. Field of the Invention

The present invention relates generally to patient monitoring, and more specifically to patient monitoring to measure signals from the patient and transmit the signals wirelessly. Although specific reference is made to patient monitoring in a hospital, embodiments of the present invention will find many applications outside the hospital, for example in home patient monitoring.

Patients are often treated for diseases and/or conditions associated with a compromised status of the patient, for example a compromised physiologic status. In some instances, a patient may report symptoms that require diagnosis and/or hospitalization to determine and treat the underlying cause. For example, a patient may have suffered a heart attack and require hospitalization and/or surgery for treatment.

Work in relation to embodiments of the present invention suggests that known methods and apparatus for monitoring of patients may be less than ideal. For example, in the intensive care unit (hereinafter ICU) and trauma unit of the hospital setting, patients can have many tubes and wires extending from the patient to machines that can make access to the anterior of the patient difficult. Also, connection of these tubes and wires to the patient can inhibit patient mobility, for example when the patient is moved for treatment and/or diagnosis. In an extreme case, for example, a patient may have an intubation tube for breathing, a naso-gastro feeding tube, separate catheters in the neck and clavicle, electrodes positioned on the chest, and probes coupled to the fingers, arms and or legs, such as blood pressure and/or oxygen measurement. These catheters, tubes and fiber optics may be connected to ventilator machines, dialysis machines, ECG machines, EEG machines, and blood pressure and oxygen monitors, and additional machines that the patient may require for life support.

Although the life support and monitoring tubes, electrodes, wires and additional equipment can help to save the patient's life, in some instances these device can also interfere with treatment of the patient. For example, it may be desirable to have access to the front of the patient without inhibition of tubes and wires, for example during open heart surgery with an anterior incision into the patient. Also, it may be necessary to move the patient, for example for imaging studies such as MRI, which require that at least some of the life support equipment move with the patient. Also, if the patient starts to seizure, for example due to central nervous system complications, the tubes wires and electrodes may shake with the patient and possibly interfere with treatment and/or diagnosis in at least some instances.

Therefore, a need exists for improved patient monitoring. Ideally, such improved patient monitoring would avoid at least some of the short-comings of the present methods and devices.

2. Description of the Background Art

Prior U.S. patents and publications describing patient monitoring include: 2007/0027388; 2006/0155183; U.S. Pat. Nos. 7,136,703; 6,814,706; 6,295,466; 5,634,468; 5,511,553; and 4,681,118.

### BRIEF SUMMARY OF THE INVENTION

Embodiments of the present invention provide devices, systems and methods for monitoring a patient from the back of the patient. The device may comprise a support configured to adhere to the back of the patient, at least two electrodes supported with the support, circuitry coupled to the at least two electrodes to measure a signal from the at least two electrodes, and circuitry to transmit the signal wirelessly. The support and the at least two electrodes may be placed on at least one of a lower back or between shoulder blades of the patient, which can help to reduce pressure on the patient when the device is worn for an extended period, for example 1 week. In addition, placement of the adherent device in at least one of these locations can improve patient comfort, for example by decreasing pressure to the skin of the patient from the device when the patient lies supine. Further, the device can provide lumbar support, for example when placed on the lumbar of the patient.

In a first aspect, embodiments of the present invention provide a method of monitoring a patient having a back and a spine disposed along a midline of the patient. A first electrode and a second electrode are placed on the back of the patient. A signal is measured from the first electrode and the second electrode.

In many embodiments, the first electrode and the second electrode are placed such that the first electrode is placed on a first side of the midline and the second electrode is placed on a second side of the midline opposite the first side. In some embodiments, the first electrode and the second electrode may be placed along the midline and aligned with the spine.

In many embodiments, the first electrode and the second electrode are placed on at least one of a lower back or between shoulder blades of the patient. The first electrode can be placed a first distance from the midline of the patient and the second electrode can be placed a second distance from the midline of the patient, such that the first distance is substantially similar to the second distance and such that the first electrode and the second electrode are symmetrically disposed on opposite sides of the midline of the patient.

In many embodiments, the signal comprises at least one of an electrocardiogram signal or a bioimpedance signal. The signal may comprise the bioimpedance signal and at least one of a hydration or a respiration of the patient may be determined from the bioimpedance signal.

In many embodiments, the first electrode and the second electrode are supported with a flexible adherent device comprising a support adhered to the back of the patient.

The support may comprise a midline and the midline of the support can be aligned with the midline of the patient when the support is adhered to the back of the patient. The support may support rigid circuitry components disposed on each side of the support away from the midline of the support so as to minimize pressure to the spine of the patient when the patient is placed in the supine position. The rigid circuitry components may comprise at least one of an integrated circuit or a rigid printed circuitry board.

In many embodiments, the device is adhered to at least one of a lower back of the patient or an upper back of the patient. The flexible adherent device can be adhered to a lower back of the patient and at least partially supports a lumbar of the spine of the patient when the patient sits and/or is placed in a supine position. The support can be adhered to the upper back between shoulder blades of the patient.

In many embodiments, the flexible adherent device comprises a central portion supported on the back with the skin of the patient, the central portion comprising a midline aligned with the midline of the patient. The flexible adherent device may comprise peripheral portions adhered to the back and extending from the central portion. The central portion may comprise a thickness of no more than about 10 mm from an inner surface configured to adhere to the skin to an outer surface opposite the inner surface, and the peripheral portions may each comprise a thickness of no more than about 5 mm from the inner surface configured to adhere to the skin of the patient to the outer surface opposite the inner surface. The thickness of the central portion may comprise no more than about 5 mm and the thickness of each peripheral portion comprises no more than about 3 mm. The central portion may comprise the midline aligned with the spine, and the peripheral portions may comprise the electrodes and extend along at least one of trapezius muscles or latissimus dorsi muscles of the patient.

In many embodiments, a second signal is measured and comprises at least one of an activity signal, a posture signal, a temperature signal or an oxygen saturation signal.

In many embodiments, the signal is transmitted wirelessly to a monitoring station in a hospital such that hospital personnel can monitor a status of the patient.

In many embodiments, an algorithm is configured to determine a condition of the patient in response to the signal and generate an alarm in response to the condition. The condition comprises an arrhythmia of the patient.

In another aspect, embodiments of the present invention provide a device for monitoring a patient having a back. The device comprises a support comprising an adhesive. The support is configured to adhere to a skin of the back of the patient. At least two electrodes are supported with the support, and circuitry is supported with the support and coupled to the at least two electrodes. The circuitry is configured to measure a signal from the electrodes and transmit the signal wirelessly.

In many embodiments, the support is configured to flex and conform to the surface contour of the skin of the patient when the support is adhered to the skin, and the support comprises at least one of a breathable tape or a flex printed circuit board configured to flex with the surface contour of the skin of the patient when the support is adhered to the skin of the patient.

In many embodiments, the support comprises a midline configured for alignment with a spine of the patient. The support may comprise a visible indicia to align the support with the midline of the patient along the spine. A first portion of the circuitry may be disposed on first side of the midline and a second portion of the circuitry may be disposed on a second side of the midline opposite the first side.

In many embodiments, the device comprises a thickness profile comprising a distance extending from a lower surface of the device configured for placement against the skin of the patient to an outer surface of the device opposite the lower surface. The thickness profile comprises a first central dimension at a first location configured for placement on the midline of the patient and a second peripheral dimension at a second location configured for placement away from the midline of the patient, in which the first central dimension is greater than the second peripheral dimension. The thickness profile may comprise a third distance at a third location, in which the third location is located away from the midline and between the first location and the second location, and the third distance may be greater than the first distance and the second distance.

In many embodiments, the device comprises a cover having an outer surface, in which the cover is disposed over the electronics and supported with the support. The cover and support may comprise a central portion of the device and peripheral portions of the device, each portion may have a thickness extending from the adhesive to the outer surface of the cover, in which the central portion comprises the midline configured for alignment with the spine of the patient and has a maximum thickness no more than about 10 mm, and in which the peripheral portions extend from the central portion and have a maximum thickness of no more than about 5 mm. The cover may comprise at least one of a coating, a dip coating, a molding, a housing, a casing or a stretchable fabric. The maximum thickness of the central portion may comprise no more than about 5 mm and the maximum thickness of each peripheral portion may comprise no more than about 3 mm. The maximum thickness of the central portion is disposed away from a midline of the central portion and wherein the central portion comprises a second thickness along the midline less than the maximum thickness of the central portion to decrease pressure on a spine of the patient when the patient lies in a supine position.

In many embodiments, the support comprises a midline and extends away from the midline symmetrically to an outer boundary disposed symmetrically about the midline, and the at least two electrodes are positioned on the support symmetrically about the midline such that the electrodes are positioned on opposite sides of the spine at equal distances from the midline when the supported is adhered to the back of the patient and the midline of the support is aligned with the spine of the patient.

In many embodiments, the support comprises at least one of a breathable tape or a flex printed circuit board configured to stretch with the skin of the patient.

In many embodiments, the support is shaped for lumbar support of the patient.

In another aspect, embodiments of the present invention provide a system for monitoring a patient having a back. The system comprises at least one support comprising an adhesive, and the at least one support is configured to adhere to a skin of the back of the patient. At least two electrodes are supported with the at least one support. Circuitry is supported with the at least one support and coupled to the at least two electrodes, and the circuitry configured to measure a signal from the electrodes and transmit wirelessly the signal from the electrodes. A gateway is configured to receive the signal from the circuitry.

In many embodiments, the circuitry is configured to monitor and transmit wirelessly to the gateway at least one of an electrocardiogram signal, a respiration rate signal, body fluid signal, an activity signal, a posture signal, a temperature signal or an oxygen saturation signal.

In many embodiments, the system further comprises at least one processor comprising a tangible medium configured to receive the signal from the gateway, and a display located at a station to monitor the patient and coupled to the at least one processor to display the signal.

In many embodiments, the at least one support comprises a first support and a second support, and the circuitry comprises first circuitry supported with the first support and second circuitry supported with the second support, the first circuitry configured to measure a first signal from the patient, the second circuitry configured to measure a second signal from the patient, the first circuitry and the second circuitry each configured to transmit signals to the gateway, the first support configured to adhere to at least one of a lower back of the patient or between shoulder blades of the patient. The first circuitry can be coupled to a first at least two electrodes configured to measure the first signal comprising a first cardiac vector in a direction extending between the first at least two electrodes, and the second circuitry can be coupled to a second at least two electrodes configured to measure the second signal comprising a second cardiac vector in a direction extending between the second at least two electrodes. The first at least two electrodes may extend substantially laterally across the back of the patient, and the second at least two electrodes may extend substantially vertically along at least one of the back or the side of the patient.

### BRIEF DESCRIPTION OF THE DRAWINGS

FIG. 1A shows a patient and a monitoring system comprising an adherent device placed on the back of a patient, according to embodiments of the present invention;

FIG. 1A1 shows a cross-sectional view of the patient with an adherent device as in FIG. 1A adhered to and conforming with the lower back of the patient;

FIG. 1A2 shows a cross-sectional view as in FIG. 1A1 with rigid components of the adherent device disposed away from a midline of the adherent device and patient so as to decrease loading of the spinous process with the rigid components of the adherent device;

FIG. 1A3 shows a dorsal view with adherent device placement and muscle groups of the patient as in FIG. 1A;

FIG. 1A3-1 shows a side view of adherent device placement and vertebrae of the patient as in FIG. 1A;

FIG. 1A4 shows an adherent device system comprising a plurality of adherent devices simultaneously adhered to the patient with at least one adherent device adhered to the back of the patient, according to embodiments of the present invention;

FIG. 1A4-1 shows sensor and circuitry of the plurality of adherent device as in FIG. 1A4;

FIG. 1A4-2 shows circuitry and sensors of an adherent device which may comprise a wireless communication circuitry, at least one battery, a processor, and an arterial blood pressure sensor and associated circuitry to amplify the arterial blood pressure signal for digitization with processor, according to embodiments;

FIG. 1A4-3 an circuitry and sensors of an adherent device which may comprise a wireless communication circuitry, at least one battery, a processor, and a pulsed oximeter sensor and associated circuitry; according to embodiments;

FIG. 1A5 shows an adherent device system comprising a first adherent device to measure a first cardiac vector and a second adherent device to measure a second cardiac vector simultaneously adhered to the patient with the first adherent device adhered to the back of the patient and the second adherent device adhered to the side of the patient, according to embodiments of the present invention;

FIG. 1B shows a bottom view of the adherent device as in FIG. 1A;

FIG. 1B1 shows a bottom view of an adherent patch similar to the patch of FIG. 1B and comprising at least four electrodes for measuring impedance, according to embodiments of the present invention;

FIG. 1C shows a top view of the adherent patch, as in FIG. 1B;

FIG. 1D shows a printed circuit boards and electronic components over the adherent patch, as in FIG. 1C;

FIG. 1E shows batteries positioned over the printed circuit board and electronic components as in FIG. 1D;

FIG. 1F shows a top view of an electronics housing and a breathable cover over the batteries, electronic components and printed circuit board as in FIG. 1E;

FIG. 1G shows a side view of the adherent device as in FIGS. 1A to 1F;

FIG. 1H shown a bottom isometric view of the adherent device as in FIGS. 1A to 1G;

FIGS. 1I and 1J show a side cross-sectional view and an exploded view, respectively, of the adherent device as in FIGS. 1A to 1H;

FIG. 1K shows at least one electrode configured to electrically couple to a skin of the patient through a breathable tape, according to embodiments of the present invention;

FIG. 2A shows a method of monitoring and/or treating a patient, according to embodiments of the present invention;

FIG. 3A shows electrode positioning for experiments measuring ECG signals, according to embodiments of the present invention;

FIG. 3B show ECG signals with electrode positions as in FIG. 3A;

FIG. 3C shows electrode positioning for experiments measuring ECG signals, according to embodiments of the present invention; and

FIG. 3D show ECG signals with electrode positions as in FIG. 3C.

### DETAILED DESCRIPTION OF THE INVENTION

An adherent cardiac monitoring system can be used for in-hospital arrhythmia detection, and may also be used for at home patient monitoring. The device can be designed to have a low profile, and the majority of the thickness of the patch can be concentrated in a central portion which can be placed on the small of the back or between the shoulder blades, where the device will minimally affect the patient's comfort such as when the patient lies supine on his or her back. With in hospital use, the wireless adherent component can be configured to be placed on the patient's back, where the device can remain out of the way of hospital personnel during monitoring. For example, the device can be placed upon the patient's back upon admission and kept out of the way of subsequent medical procedures. Although the adherent device can be used in a hospital setting, it may be used for in home monitoring where the placement of the device on the back of the patient can minimize interference with the day to day activities of the patient.

The adherent devices described herein may be used for 90 day monitoring, or more, and may comprise completely disposable components and/or reusable components, and can provide reliable data acquisition and transfer. In many embodiments, the patch is configured for patient comfort, such that the adherent patch can be worn and/or tolerated by the patient for extended periods, for example 90 days or more. The patch may be worn continuously for at least seven days, for example 14 days, and then replaced with another patch. Adherent devices with comfortable patches that can be worn for extended periods and in which patches can be replaced and the electronics modules reused. In many embodiments, the adherent patch comprises a tape, which comprises a material, preferably breathable, with an adhesive, such that trauma to the patient skin can be minimized while the patch is worn for the extended period. The printed circuit board may comprise a flex printed circuit board that can flex with the patient to provide improved patient comfort.

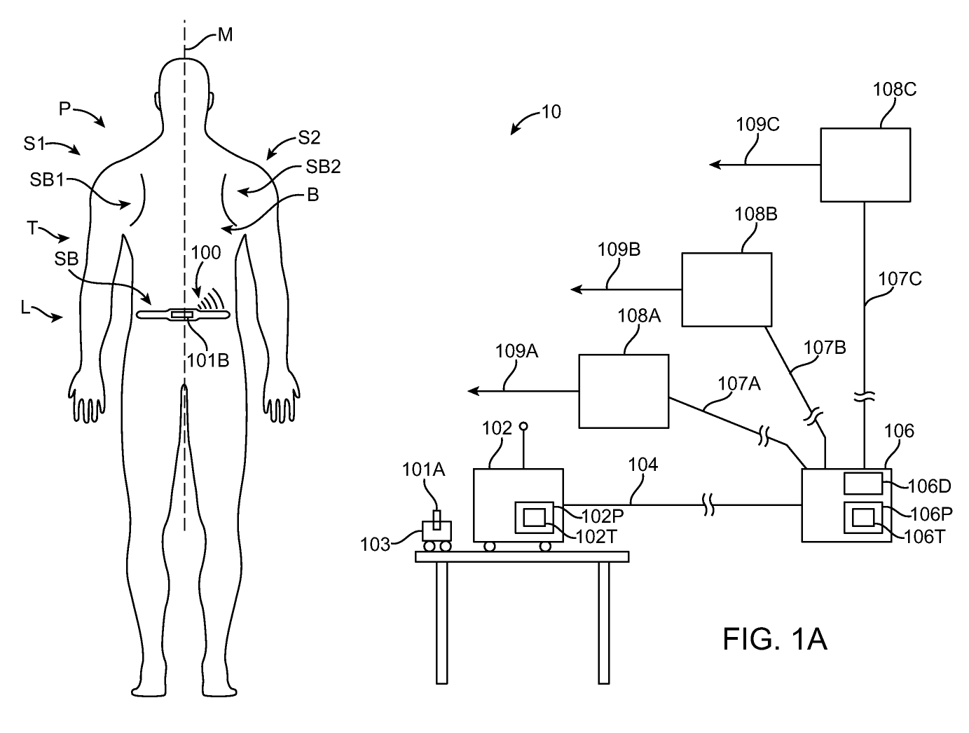

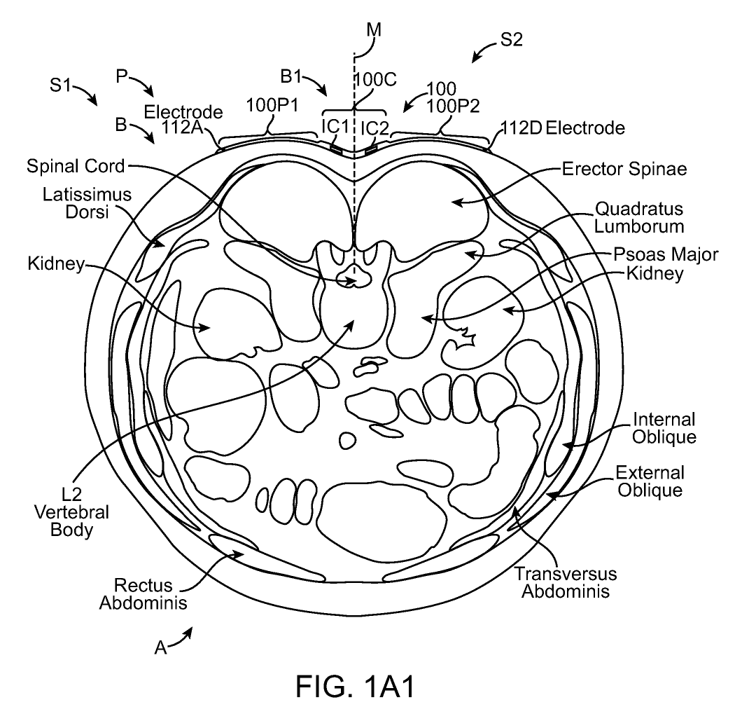

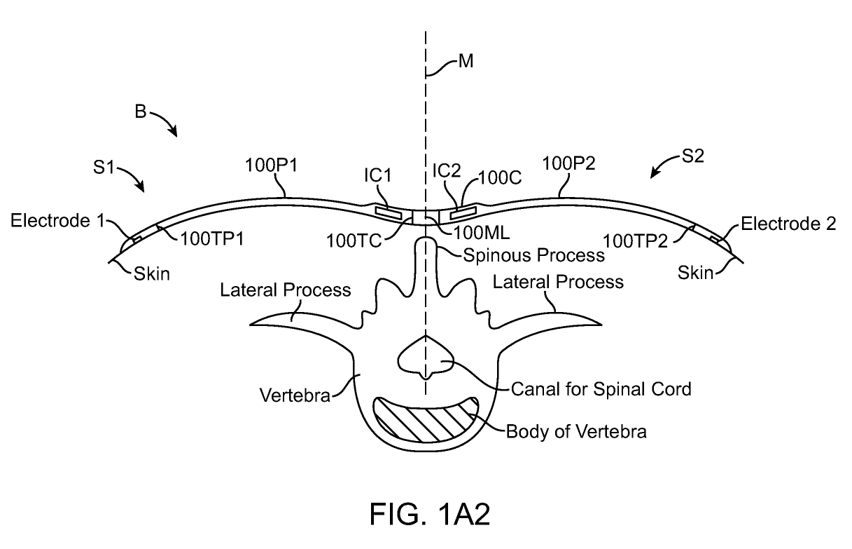

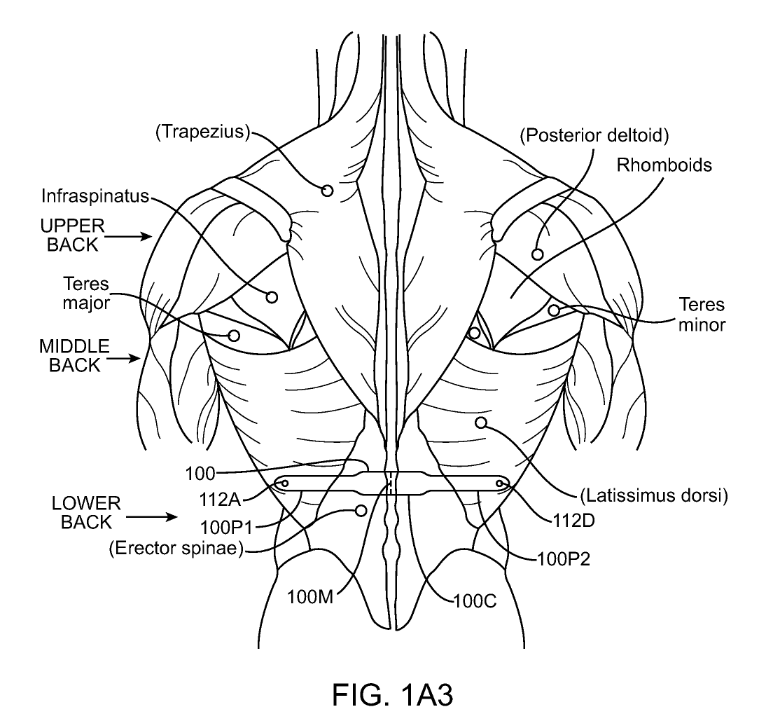

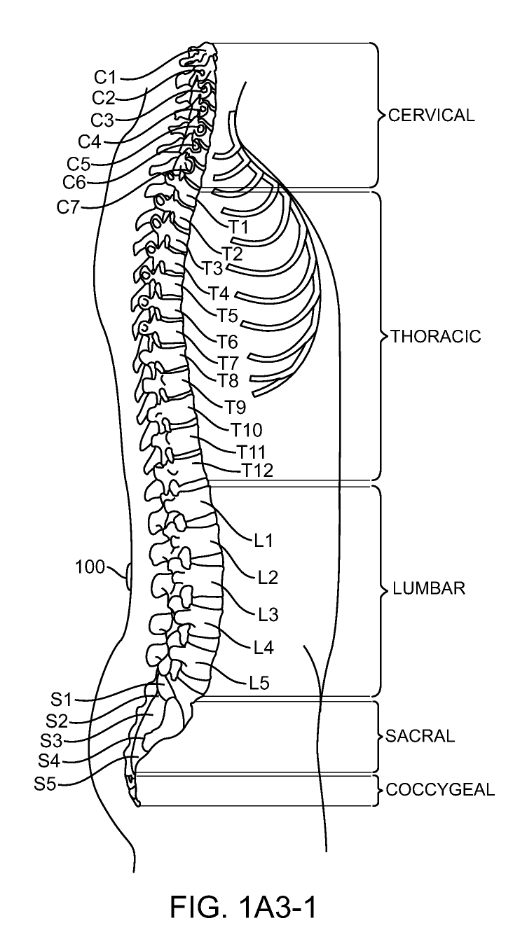

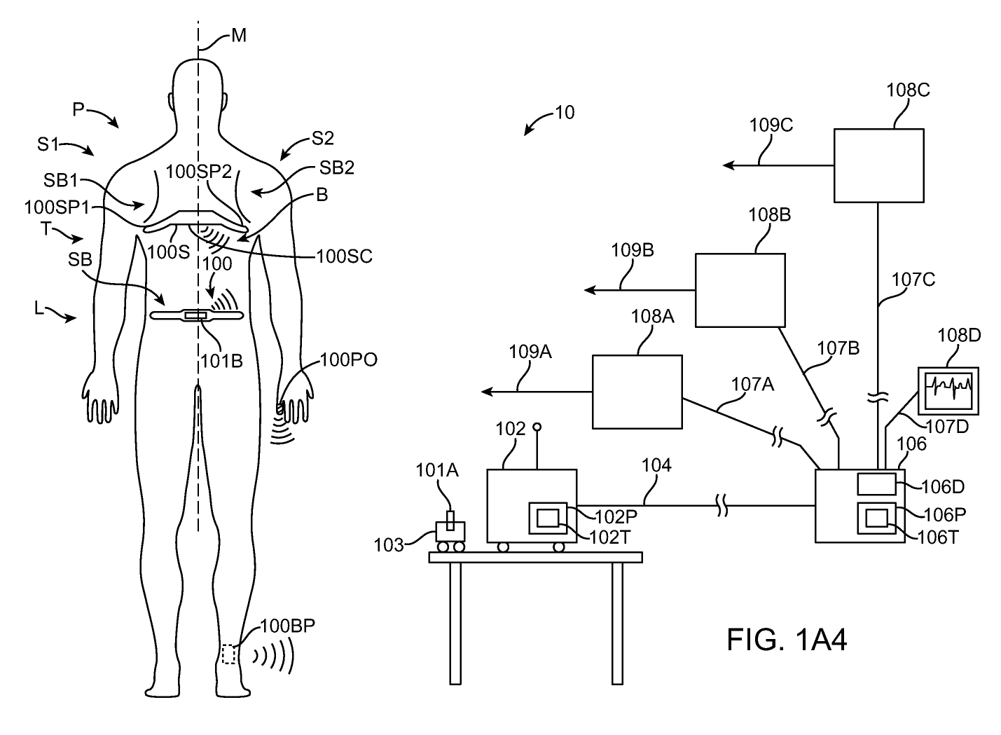

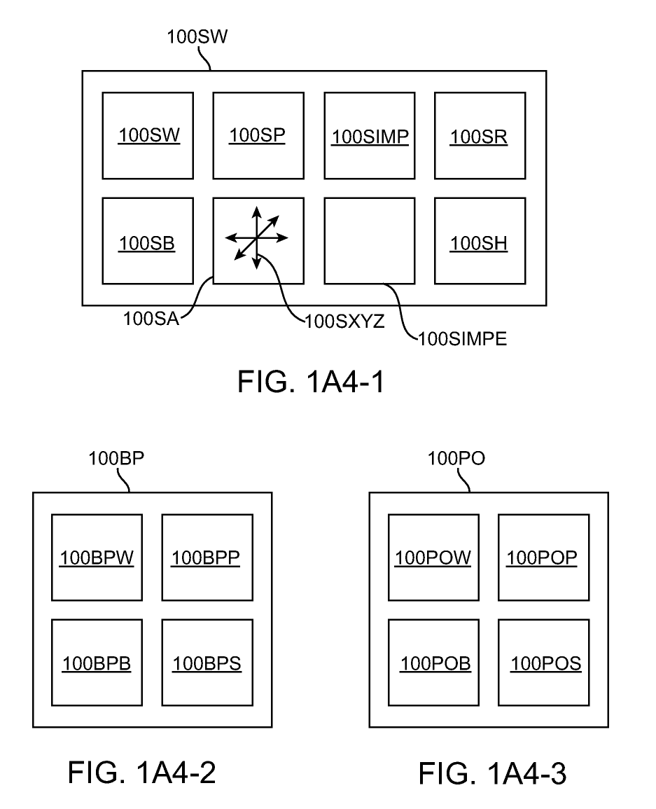

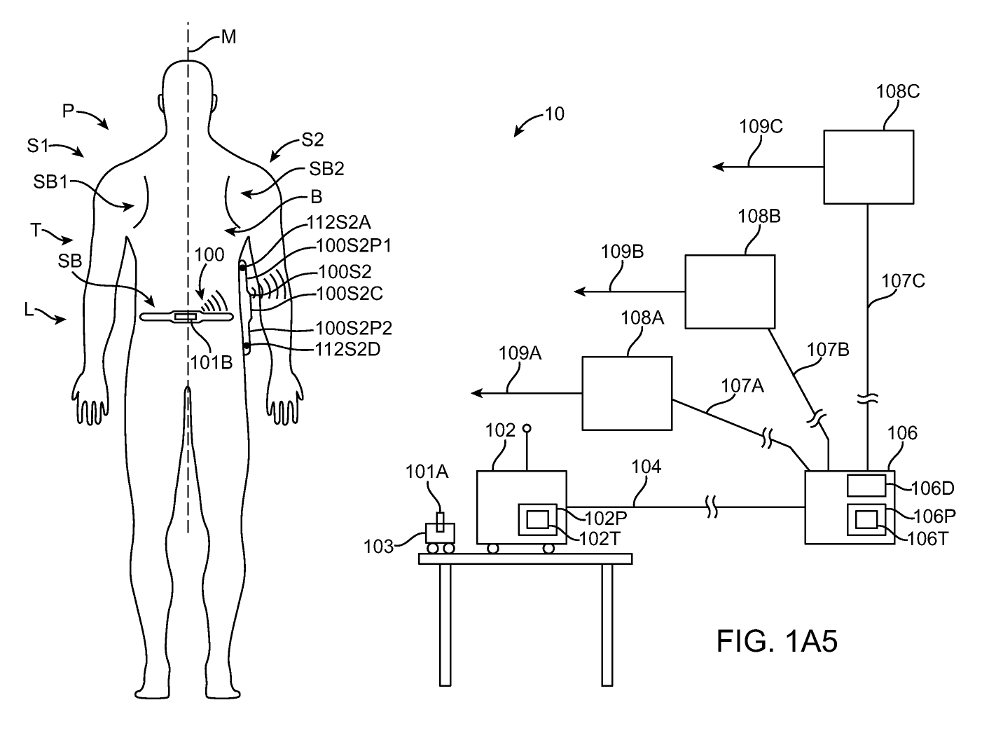

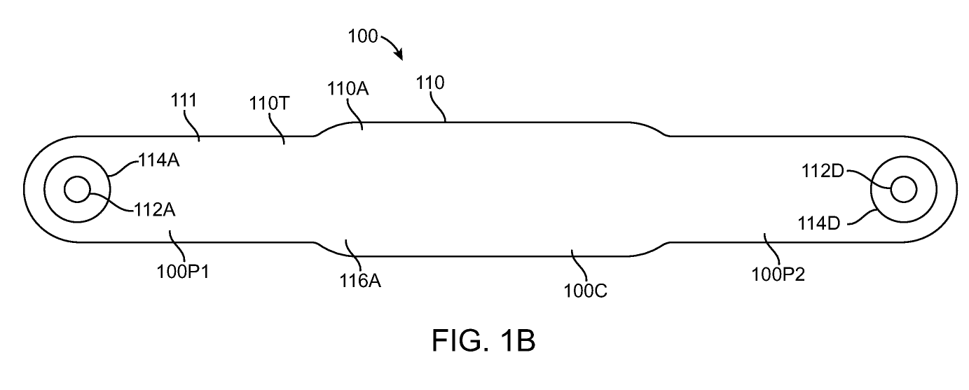

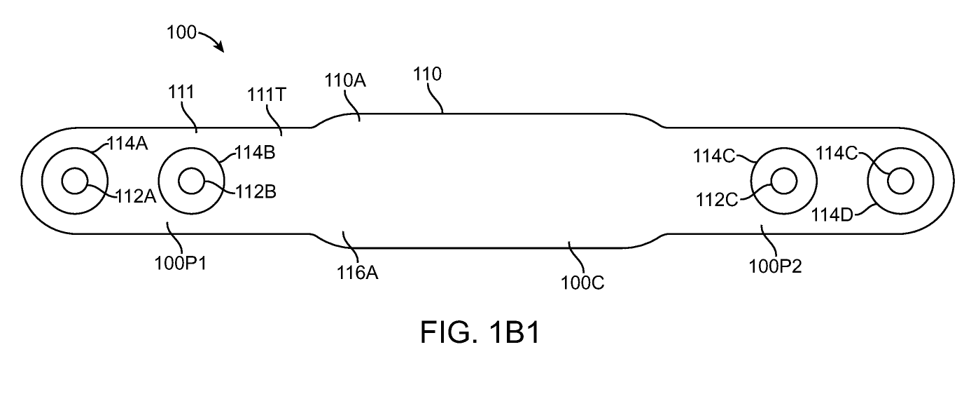

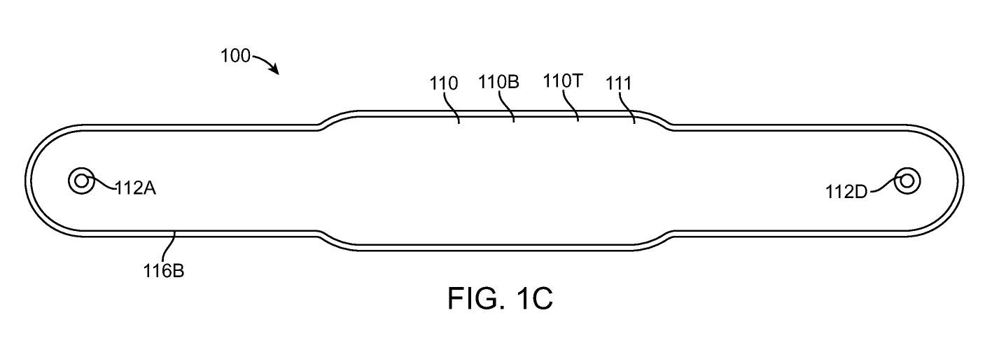

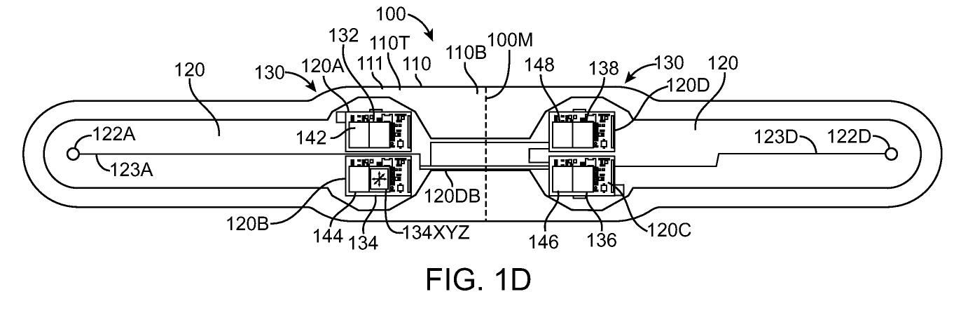

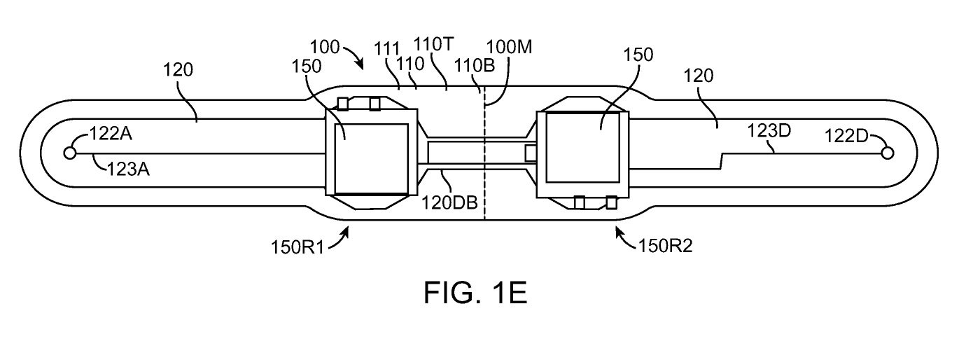

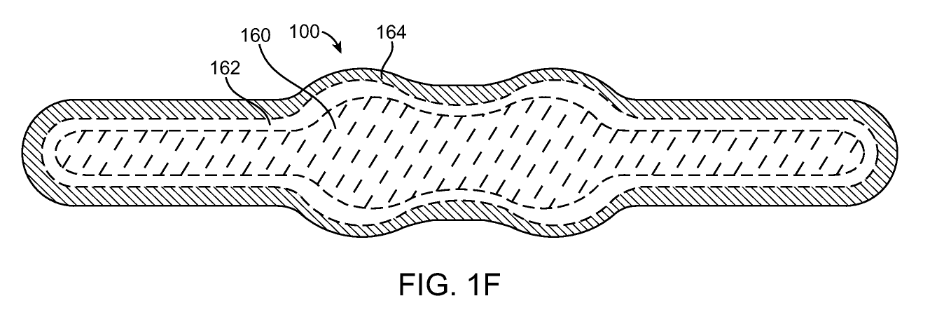

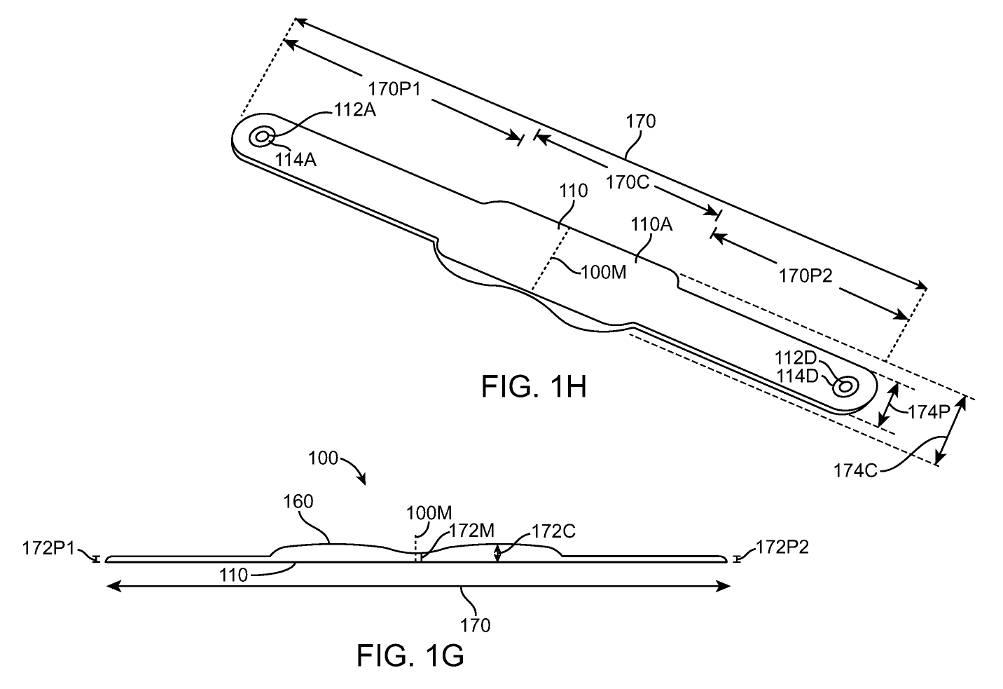

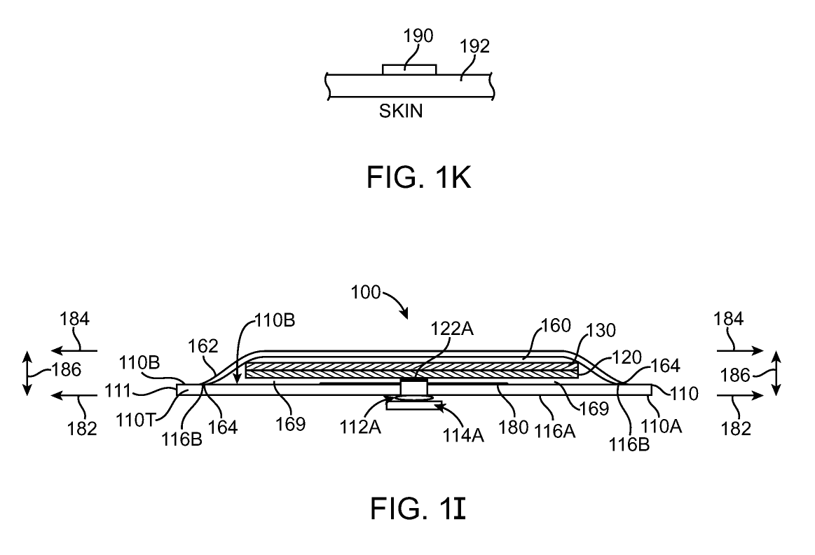

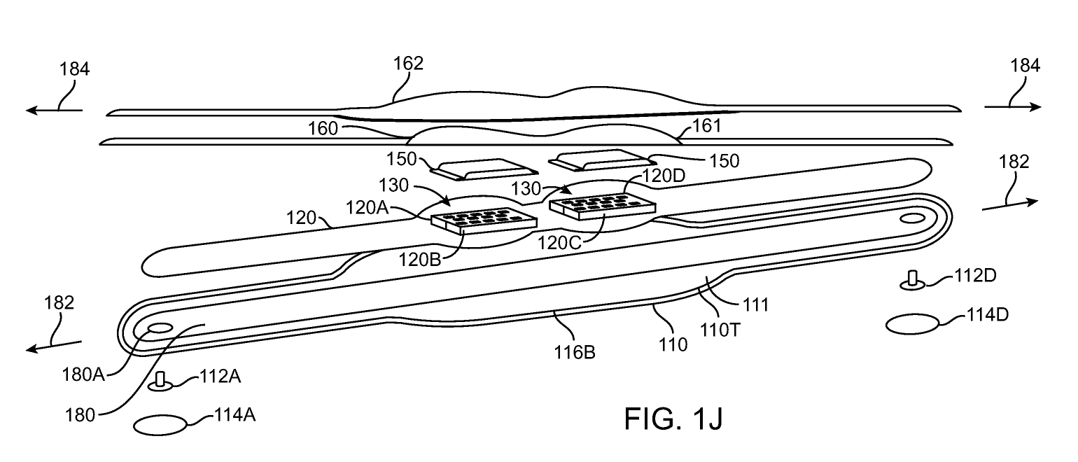

FIG. 1A shows a patient P and a monitoring system 10. Patient P comprises a midline M, a first side S1, for example a left side, and a second side S2, for example a right side. Monitoring system 10 comprises an adherent device 100. Adherent device 100 can be adhered to a patient P at many locations, for example thorax T of patient P. In many embodiments, the adherent device may adhere to symmetrically about a midline of the patient, from which location data can be collected. Work in relation with embodiments of the present invention suggests that location on the back the patient, for example the small of the back, or lower back, over lumbar vertebrae and/or between shoulder blades, can provide comfort for the patient when the device is adhered to the patient. Another useful location to place the device is over the thoracic vertebrae, for example with the central portion of the device flexible and sized to fit near the spine and a thinner peripheral portion sized to extend over and flex with the ribs. Such placement on the thoracic vertebrae can be helpful, particularly when the patient is mobile. Placement on the small of the back, or lower back, can minimize pressure to the device and patient when the patient lies supine on his or her back. Further, placement of the device on the lower back over the lumbar vertebrae can provide support to the lumbar vertebra, also referred to as lumbar support, when the patient sits and/or when the patient lies supine. Similarly, placement of the adherent device between the shoulder blades over thoracic vertebrae can decrease pressure to the device and patient when the patient lies supine on his or her back.

Monitoring system 10 includes components to transmit data to a computer system 106. Computer system 106 can be located in the same building as the patient. For example, computer system 106 can be located at a central monitoring station for a ward in a hospital, for example a nurses' station. In some embodiments, for example with in home patient monitoring, computer system 106 can be located far from the patient, for example the patient located on a first continent and the computer system located on a second continent.

Adherent device 100 can communicate wirelessly to an intermediate device 102, for example with a single wireless hop from the adherent device on the patient to the intermediate device. Intermediate device 102 can communicate with computer system 106 in many ways, for example with a wireless connection 104, an intranet, an Ethernet, an internet connection and/or with a cellular connection. Intermediate device 102 can be located in the hospital room with the patient to receive patient data stored on the adherent device. In some embodiments, intermediate device 102 comprises a plurality of intermediate devices with a first intermediate device disposed at the hospital and a second intermediate device disposed at the patient's home. In many embodiments, monitoring system 10 comprises a distributed processing system with at least one processor comprising a tangible medium of device 100, at least one processor 102P of intermediate device 102, and at least one processor 106P of computer system 106, each of which processors can be in electronic communication with the other processors. At least one processor 102P comprises a tangible medium 102T, and at least one processor 106P comprises a tangible medium 106T. At least one processor 106P may comprise a backend server located at the computer system.

Computer system 106 may comprise a display 106D for the healthcare provider to view patient data, for example for the nurses to view heart rate signals measured from the patient. Display 106D can be located in the hospital at the nurses' station to allow doctors, nurses and technicians to view patient data when treating the patient. In some embodiments, the patient information can be sent to the health care provider at a location remote from the patient, for example when the patient and health care provider are located in separate buildings. Patient data can be sent to a handheld device to allow remote treatment of the patient.

Computer system 106 can be in communication with a health care provider 108A with a communication system 107A, such as a page, the Internet, an intranet, phone lines, wireless and/or satellite phone. Health care provider 108A, for example a doctor's assistant, can treat patient P as indicated by arrow 109A in response to alerts from the processor system. Computer system 106 can be in communication with a health care professional, for example a doctor 108B, with a communication system 107B, similar to communication system 107A, and coupled with a handheld device, such as the Internet, an intranet, phone lines, wireless and/or satellite phone. Doctor 108B can be in communication with patient P and/or provider 108A with a communication system comprising a handheld device, for example with a two way communication system, as indicated by arrow 109B, for example by cell phone, email, landline. Thus, in many embodiments, monitoring system 10 comprises a closed loop system in which patient care can be monitored and implemented from the computer system in response to signals from the adherent device. With in home monitoring, computer system 106 can communicate with a family member 108C with communication system 107C, similar to communication system 107C. The family member can respond in response to a signal from computer system 106, for example a notification and/or alert and attend to the patient.

In many embodiments, computer system 106 receives the patient data and applies a patient evaluation algorithm, for example an algorithm to detect cardiac arrhythmia from an electrocardiogram signal of the adherent device. Computer system 106, and/or the processor of the adherent device, can determine the heart rate variability in many ways, for example with at least one of time domain determination, frequency domain determination or non-linear determination.

The adherent device may be affixed and/or adhered to the body in many ways. For example, with at least one of the following: an adhesive tape, a constant-force spring, suspenders around shoulders, a screw-in microneedle electrode, a pre-shaped electronics module to shape fabric to a thorax, a pinch onto roll of skin, or transcutaneous anchoring. Patch and/or device replacement may occur with a keyed patch (e.g. two-part patch), an outline or anatomical mark, a low-adhesive guide (place guide|remove old patch|place new patch|remove guide), or a keyed attachment for chatter reduction. The patch and/or device may comprise an adhesiveless embodiment (e.g. waist strap), and/or a low-irritation adhesive for sensitive skin. The adherent patch and/or device can comprise many shapes, for example at least one of a dogbone, an hourglass, an oblong, a circular or an oval shape.

In many embodiments, the adherent device may comprise a reusable electronics module with replaceable patches, and each of the replaceable patches may include a battery. The module may collect cumulative data for approximately 90 days and/or the entire adherent component (electronics+patch) may be disposable. In a completely disposable embodiment, a “baton” mechanism may be used for data transfer and retention, for example baton transfer may include baseline information. In some embodiments, the device may have a rechargeable module, and may use dual battery and/or electronics modules, wherein one module 101A can be recharged using a charging station 103 while the other module 101B is placed on the adherent patch with connectors. In some embodiments, the intermediate device 102 may comprise the charging module, data transfer, storage and/or transmission, such that one of the electronics modules can be placed in the intermediate device for charging and/or data transfer while the other electronics module is worn by the patient.

System 10 can perform the following functions: initiation, programming, measuring, storing, analyzing, communicating, predicting, and displaying. The adherent device may contain a subset of the following physiological sensors: bioimpedance, respiration, respiration rate variability, heart rate (ave, min, max), heart rhythm, heart rate variability (HRV), heart rate turbulence (HRT), heart sounds (e.g. S3), respiratory sounds, blood pressure, activity, posture, wake/sleep, orthopnea, temperature/heat flux, and weight. The activity sensor may comprise one or more of the following: ball switch, accelerometer, minute ventilation, HR, bioimpedance noise, skin temperature/heat flux, BP, muscle noise, posture.

The adherent device can wirelessly communicate with computer system 106. The communication may occur directly (via a cellular or Wi-Fi network), or indirectly through intermediate device 102. Intermediate device 102 may consist of multiple devices, which can communicate wired or wirelessly to relay data to computer system 106.

In many embodiments, instructions are transmitted from computer system 106 to a processor supported with the adherent patch on the patient, and the processor supported with the patient can receive updated instructions for the patient treatment and/or monitoring, for example while worn by the patient.

FIG. 1A1 shows a cross-sectional view of the patient P with adherent device 100 as in FIG. 1A adhered to and conforming with back B of the patient. Adherent device 100 is aligned with midline M of the patient. The contour of the skin near midline M of patient P shows an indentation BI on the midline that extends laterally toward the sides of the patient. Device 100 comprises a central portion 100C sized to fit in indentation BI of the back, for example at the small of the back over lumbar vertebrae. Central portion 100C is sized to fit on the midline over the erector spinae of the patient and comprises a flexible support such that central portion 100C can flex to conform the indentation of the back BI. Device 100 comprises a first peripheral portion 100P1, or first wing, and a second peripheral portion 100P2, or second wing, each of which extends from central portion 100C. First peripheral portion 100P1 and second peripheral portion 100P2 may each have a thickness no more than about 5 mm thick, so as to decrease pressure to the skin of the patient. The central portion and the peripheral portions can each flex, for example with a flexible support, so as to conform to the back of the patient. The peripheral portions and central portion can be configured to flex with the patient and many comprise a flex PCB with traces that extend to electrodes.

Because placement of electrodes on the back can result in measurement of the cardiac signal through the tissues near the back of the patient, it can be helpful to increase the separation distance between electrode 112A and electrode 112D, as this increase in separation distance can increase the amplitude of the cardiac signal measured. Such an increase in distance can also be helpful with patient impedance measurements. Therefore, the peripheral portions can extend beyond the indentation of the back to measure signals, for example to extend over the latissimus dorsi muscles.

FIG. 1A2 shows a cross-sectional view as in FIG. 1A1 with rigid components of the adherent device disposed away from a midline of the adherent device and patient so as to decrease loading of the spinous process. This placement of the rigid components on central portion 100C away from the midline and the spinous process can minimize patient discomfort. Rigid components of device 100 may comprise at least one of an integrated circuit, a rigid printed circuitry board, or a battery. Device 100 comprises first integrated circuit IC1 disposed of side S1 of patient P and second integrated circuit IC2 disposed on second side S2 of patient P. The rigid components, for example first integrated circuit IC1 and a second integrated circuit IC2 can be positioned away from the midline to decrease loading to the spinal process that can extend toward the skin of the patient. As the spinous process can extend toward the skin of the patient and can be less conforming than adjacent tissues such as the erector spinae muscles, placement of the rigid components away from the midline can decrease loading on the spinous process from the rigid component and may decrease skin irritation.

FIG. 1A3 shows a dorsal view with patch placement and muscle groups of the patient as in FIG. 1A. Adherent device 100 is shown positioned on the lower back with central portion 100C positioned over the erector spinae muscles. The peripheral portion extends over the latissimus dorsi muscles to measure ECG signals with the electrodes. In some embodiments, the central portion can be positioned between the shoulder blades of the patient.

FIG. 1A3-1 shows a side view of placement of adherent device 100 in relation to vertebrae of the patient as in FIG. 1A. The spine of the patient includes cervical vertebrae C1-C7, Thoracic vertebra T1-T12, lumbar vertebra L1-L5 and sacral vertebrae S1-S5. Device 100 is shown placed on the small of the back substantially over lumbar vertebra L3. This side profile view shows that placement of device 100 on the back at a location between about T7 and S1 can provide decrease pressure on the skin of the patient when the patient lies supine, for example at a location between about T10 and S1. Placement of device 100 over one or more of the Lumbar vertebra L1-L5, for example L3, can provide lumbar support, for example when the patient lies supine, for example on his or her back in bed.

FIG. 1A4 shows an adherent device system 100S comprising a plurality of adherent devices simultaneously adhered to the patient, for example adherent device 100, second adherent device 100S, third adherent device 100BP and fourth adherent device 100PO. Adherent device system 100S may comprise wireless communication between and/or among devices adhered to the patient. Adherent device system 100S may comprise a component of system 10 described above. Second adherent device 100S can be positioned between the shoulder blades of the patient. Second adherent device 100S may comprise a central portion 100SC and a first peripheral portion 100SP1, or first wing, and a second peripheral portion 100SP2, or second wing, each extending peripherally from central portion 100SC. Second adherent device 100S, for example central portion 100SC, may comprise an accelerometer such as a position sensitive 3D accelerometer to generate an accelerometer signal so as to detect patient head orientation and/or movement. Second adherent device 100S may comprise electrodes on the peripheral portions for measuring ECG data and/or impedance data such as respiration and/or hydration. Third adherent device 100A may comprise a blood pressure sensor, for example an arterial blood pressure sensor coupled to an artery and disposed near an ankle, leg or foot of the patient. Third adherent device 100BP can measure blood pressure to generate a blood pressure signal and transmit the blood pressure signal wirelessly to the intermediate device and display 106D. Fourth adherent device 100PO may comprise an oxygen sensor such as a pulsed oximeter to measure patient blood oxygen levels and generate an oxygen signal. Fourth adherent device 100PO may transmit the oxygen signal wirelessly to the intermediate device and display 106D. A display 108D can be located in the patient's room to display the patient data from the sensors transmitted wirelessly, for example to display at least one of an ECG signal, a respiration signal, a hydration signal, a blood pressure signal, or a blood oxygen signal.

One will appreciate that surgery can be performed on patient P with at least one adherent device adhered to the back of the patient. The patient can lie on his or her back when the surgery is performed such that the at least one adherent device has minimal interference with the surgery to the patient. The surgery may comprise surgery with anterior access through the skin on the front of the patient while the patient lays spine. The adherent device can be adhered to the patient prior to making the incision, for example when the patient is admitted to the hospital. The at least one adherent device can also be helpful for patients who are very sick and can allow the patient to lie on his or her back for extended periods with minimal interference from the at least one adherent device.

FIG. 1A4-1 shows detail of second adherent device 100S. Second adherent device 100S may comprise a wireless communication circuitry 100SW, at least one battery 100SB, a processor 100SP, an accelerometer 100SA, impedance electrodes 100IMPE and impedance circuitry 100IMPC. Accelerometer 100SA may comprise a 3D accelerometer 100SXYZ sensitive to gravity and configured to generate an accelerometer signal so as to measure at least one of patient position or patient inclination. Impedance electrodes 100IMPE can be connected to the patient with the peripheral portions of device 100SP. For example first peripheral portion 100SPI may comprise an outer drive electrode to pass current and an inner sense electrode to measure voltage, and second peripheral portion 100SP2 may comprise an outer drive electrode to pass current and an inner sense electrode to measure voltage. Impedance circuitry 100SIMP can be connected to impedance electrodes 100IMPE to measure impedance signals of the patient, for example four pole impedance measurements with two inner sense electrodes and two outer drive electrodes. Circuitry 100SR can determine the respiration rate of the patient from the impedance signal, and circuitry 100SH can measure hydration of the patient from the impedance signal. Processor 100SP comprises an tangible medium and can be configured process signals and/or data from the accelerometer and/or impedance circuitry. In some embodiments, processor 100SP comprises respiration circuitry 100SR and hydration circuitry 100SH. Wireless communication circuitry 100SW can transmit the data to other components of system 10, for example device 100 and/or intermediate device 102.

FIG. 1A4-2 shows detail of third adherent device 100BP. Third adherent device 100BP may comprise a wireless communication circuitry 100BPW, at least one battery 100BPB, a processor 100BPP and an arterial blood pressure sensor 100BPS and associated circuitry to amplify the arterial blood pressure signal for digitization with processor 100BPP. Blood pressure sensor 100BPS may comprise known blood pressure sensors and configured to generate a blood pressure signal so as to measure the blood pressure at the artery of the patient. Processor 100BPP can process signals and/or data from the blood pressure sensor. Wireless communication circuitry 100BPW can transmit the data to other components of system 10, for example device 100 and/or intermediate device 102.

FIG. 1A4-3 shows detail of third adherent device 100PO. Third adherent device 100PO may comprise a wireless communication circuitry 100POW, at least one battery 100POB, a processor 100POP and a pulsed oximeter sensor 100POS and associated circuitry. Pulsed oximeter 100POS may comprise a known pulsed oximeter sensor, circuitry, and optical apparatus for determining patient oxygen. Processor 100POP can process signals and/or data from the pulsed oximeter sensor and circuitry. Wireless communication circuitry 100POW can transmit the data to other components of system 10, for example device 100 and/or to intermediate device 102.

FIG. 1A5 shows an adherent device system comprising a first adherent device, such as device 100, to measure a first cardiac vector and a second adherent device 100S2 to measure a second cardiac vector simultaneously adhered to the patient with the first adherent device adhered to the back of the patient and the second adherent device adhered substantially vertically to at least one of the side or the back of the patient. Second adherent device 100S2 may comprise structures and circuitry substantially similar to adherent device 100, as described above. For example, second adherent device 100S2 may comprise a central portion 100S2C, a first peripheral portion 110S2P1 and a second peripheral portion 100S2P2, substantially similar to the central, first peripheral portion and second peripheral portion, respectively, as described above. Second adherent device 100S2 may comprise a first electrode 112S2A and a second electrode 112S2D, for example substantially similar to electrode 112A and electrode 112D as described above. Second adherent device 100S2 may comprise wireless circuitry, processor circuitry and many of the components described above with reference to device 100. In many embodiments, the first adherent device comprising device 100 measures a electrocardiogram vector along a first direction, for example extending between electrode 112A and electrode 112D, and the second adherent device measures the electrocardiogram vector along a second direction, for example extending between electrode 112S2A and electrode 112S2D. The first direction can be different than the second direction. For example the first direction can extend substantially laterally across the back of the patient and the second direction can extend substantially vertically along at least one of the back or the side of the patient, such that the first direction is substantially perpendicular to the second direction.

As explained below with reference to FIGS. 1B to 1J, device 100 may comprise many sensors to measure the patient from one adherent device, for example adhered to the lower back and aligned to the midline of the patient.

FIG. 1B shows a bottom view of adherent device 100 as in FIG. 1A comprising an adherent patch 110. Adherent patch 110 comprises a first side, or a lower side 110A, that is oriented toward the skin of the patient when placed on the patient. In many embodiments, adherent patch 110 comprises a tape 110T which is a material, preferably breathable, with an adhesive 116A. Patient side 110A comprises adhesive 116A to adhere the patch 110 and adherent device 100 to patient P. Electrodes 112A and 112D are affixed to adherent patch 110. In many embodiments, at least two electrodes are attached to the patch. The patch may comprise two electrodes to measure the electrocardiogram (ECG) of the patient. Gel 114A and gel 114D can each be positioned over electrodes 112A and 112D, respectively, to provide electrical conductivity between the electrodes and the skin of the patient. In many embodiments, the electrodes can be affixed to the patch 110, for example with known methods and structures such as rivets, adhesive, stitches, etc. In many embodiments, patch 110 comprises a breathable material to permit air and/or vapor to flow to and from the surface of the skin.

FIG. 1B-1 shows a bottom view of adherent patch 110 with at least four electrodes for measuring impedance. In addition to electrodes 112A and 112D, as described above, the adherent patch may comprise electrodes 112B and 112C. Electrodes 112A and 112D may comprise outer drive electrodes to drive current through tissue. Electrodes 112B and 112C may comprises inner measurement electrodes, or sense electrodes, to measure voltage through tissue when current is passed so as to determine impedance of the tissue, for example with a four pole impedance measurement. Although four electrodes are shown, some embodiments may comprise, for example, three electrodes. Four electrodes, for example electrodes 112A, 112B, 112C and 112D, can be used to measure hydration and respiration of the patient, for example with impedance measurements. The gel 114B and gel 114C can be disposed over electrodes 112B and 112C, respectively.

FIG. 1C shows a top view of the adherent patch 100, as in FIG. 1B. Adherent patch 100 comprises a second side, or upper side 110B. In many embodiments, electrodes 112A and 112D extend from lower side 110A through adherent patch 110 to upper side 110B. An adhesive 116B can be applied to upper side 110B to adhere structures, for example a breathable cover, to the patch such that the patch can support the electronics and other structures when the patch is adhered to the patient. The PCB may comprise completely flex PCB, rigid PCB, rigid PCB combined flex PCB and/or rigid PCB boards connected by cable.

FIG. 1D shows a printed circuit boards and electronic components over adherent patch 110, as in FIG. 1A to 1C. In some embodiments, a printed circuit board (PCB), for example flex printed circuit board 120, may be connected to electrodes 112A and 112D with connectors 122A and 122D. Flex printed circuit board 120 can include traces 123A and 123D that extend to connectors 122A and 122D, respectively, on the flex PCB. Connectors 122A and 122D can be positioned on flex printed circuit board 120 in alignment with electrodes 112A and 112D so as to electrically couple the flex PCB with the electrodes. In some embodiments, connectors 122A and 122D may comprise insulated wires and/or a film with conductive ink that provide strain relief between the PCB and the electrodes. For example, connectors 122A and 122D may comprise a flexible polyester film coated with conductive silver ink. In some embodiments, additional PCB's, for example rigid PCB's 120A, 120B, 120C and 120D, can be connected to flex printed circuit board 120. The rigid PCB's and components mounted thereon are shown positioned away from a midline 100M of device 100, such that the rigid components are away from the midline of the patient when the device is adhered to the patient. Electronic components 130 can be connected to flex printed circuit board 120 and/or mounted thereon. In some embodiments, electronic components 130 can be mounted on the additional PCB's.

Electronic components 130 comprise components to take physiologic measurements, transmit data to computer system 106 and receive commands from computer system 106. In many embodiments, electronics components 130 may comprise known low power circuitry, for example complementary metal oxide semiconductor (CMOS) circuitry components. Electronics components 130 may comprise an activity sensor and activity circuitry 134, impedance circuitry 136 and ECG circuitry 136. In some embodiments, electronics circuitry 130 may comprise a microphone and microphone circuitry 142 to detect an audio signal from within the patient, and the audio signal may comprise a heart sound and/or a respiratory sound, for example an S3 heart sound and a respiratory sound with rales and/or crackles.

Electronics circuitry 130 may comprise a temperature sensor, for example a thermistor in contact with the skin of the patient, and temperature sensor circuitry 144 to measure a temperature of the patient, for example a temperature of the skin of the patient. A temperature sensor may be used to determine the sleep and wake state of the patient. The temperature of the patient can decrease as the patient goes to sleep and increase when the patient wakes up.

Electronics circuitry 130 may comprise a processor 146. Processor 146 comprises a tangible medium, for example read only memory (ROM), electrically erasable programmable read only memory (EEPROM) and/or random access memory (RAM). Processor 146 may comprise many known processors with real time clock and frequency generator circuitry, for example the PIC series of processors available from Microchip, of Chandler Ariz. In some embodiments, processor 136 may comprise the frequency generator and real time clock. The processor can be configured to control a collection and transmission of data from the impedance circuitry electrocardiogram circuitry and the accelerometer. In many embodiments, device 100 comprise a distributed processor system, for example with multiple processors on device 100.

Electronics circuitry 130 may comprise electromyogram (hereinafter “EMG”) circuitry 148 to measure muscle activity. EMG circuitry 148 can measure signals from muscles and may be connected to and/or comprise at least two of electrode 112A, electrode 112B, electrode 112C or electrode 112D. EMG circuitry 148 comprises an amplifier to amplify signals from contracting muscles so as to generate an EMG signal. EMG circuitry 148 can be connected to processor to send the EMG signal to the processor for storage and/or analysis.

In many embodiments, electronics components 130 comprise wireless communications circuitry 132 to communicate with computer system 106. The wireless communication circuitry can be coupled to the impedance circuitry, the electrocardiogram circuitry and the accelerometer to transmit to a computer system with a communication protocol at least one of the hydration signal, the electrocardiogram signal or the inclination signal. In specific embodiments, wireless communication circuitry is configured to transmit the hydration signal, the electrocardiogram signal and the inclination signal to the computer system with a single wireless hop, for example from wireless communication circuitry 132 to intermediate device 102. The communication protocol comprises at least one of Bluetooth, Zigbee, WiFi, WiMax, IR, amplitude modulation or frequency modulation. In many embodiments, the communications protocol comprises a two way protocol such that the computer system is capable of issuing commands to control data collection.

Intermediate device 102 may comprise a data collection system to collect and store data from the wireless transmitter. The data collection system can be configured to communicate periodically with the computer system. The data collection system can transmit data in response to commands from computer system 106 and/or in response to commands from the adherent device.

Activity sensor and activity circuitry 134 can comprise many known activity sensors and circuitry. In many embodiments, the accelerometer comprises at least one of a piezoelectric accelerometer, capacitive accelerometer or electromechanical accelerometer. The accelerometer may comprises a 3-axis accelerometer 134XYZ to generate an accelerometer signal so as to measure at least one of an inclination, a position, an orientation or acceleration of the patient in three dimensions. Work in relation to embodiments of the present invention suggests that three dimensional orientation of the patient and associated positions, for example sitting, standing, lying down, can be very useful when combined with data from other sensors, for example ECG data and/or bioimpedance data, for example a respiration rate of the patient.

Impedance circuitry 136 can generate both hydration data and respiration data. In many embodiments, impedance circuitry 136 is electrically connected to electrodes 112A and 112D and additional electrodes 112B and 112C, as described above, in a four pole configuration, such that electrodes 112A and 112D comprise outer electrodes that are driven with a current and comprise force electrodes that force the current through the tissue. The current delivered between electrodes 112A and 112D generates a measurable voltage between the additional electrodes 112B and 112C, such that the additional electrodes 112B and 112C may comprise inner, sense, electrodes that sense and/or measure the voltage in response to the current from the force electrodes.

ECG circuitry 138 can generate electrocardiogram signals and data from two or more of electrodes 112A and 112D in many ways, for example with an instrumentation amplifier coupled to electrodes 112A and 112D.

FIG. 1E shows batteries 150 positioned over the flex printed circuit board and electronic components as in FIG. 1D. Batteries 150 may comprise rechargeable batteries that can be removed and/or recharged. In some embodiments, batteries 150 can be removed from the adherent patch and recharged and/or replaced.

FIG. 1F shows a top view of a cover 162 over the batteries, electronic components and flex printed circuit board as in FIGS. 1A to 1E. In many embodiments, an electronics housing 160 may be disposed under cover 162 to protect the electronic components, and in some embodiments electronics housing 160 may comprise an encapsulant over the electronic components and PCB. In some embodiments, cover 162 can be adhered to adherent patch 110 with an adhesive 164 on an underside of cover 162. In many embodiments, electronics housing 160 may comprise a water proof material, for example a sealant adhesive such as epoxy or silicone coated over the electronics components and/or PCB. In some embodiments, electronics housing 160 may comprise metal and/or plastic. Metal or plastic may be potted with a material such as epoxy or silicone.

Cover 162 may comprise many known biocompatible cover, casing and/or housing materials, such as elastomers, for example silicone. The elastomer may be fenestrated to improve breathability. In some embodiments, cover 162 may comprise many known breathable materials, for example polyester, polyamide, and/or elastane (Spandex). The breathable fabric may be coated to make it water resistant, waterproof, and/or to aid in wicking moisture away from the patch.

FIG. 1G shows a side view of adherent device 100 as in FIGS. 1A to 1F. Adherent device 100 comprises a maximum dimension, for example a length 170 from about 2 to 10 inches (from about 50 mm to about 250 mm), for example from about 4 to 6 inches (from about 100 mm to about 150 mm). In some embodiments, length 170 may be no more than about 6 inches (no more than about 150 mm). Adherent device 100 comprises a thickness 172. Thickness 172 may comprise a maximum thickness along a profile of the device. Thickness 172 can be from about 0.2 inches to about 0.4 inches (from about 5 mm to about 10 mm), for example about 0.3 inches (about 7.5 mm).

FIG. 1H shown a bottom isometric view of adherent device 100 as in FIGS. 1A to 1G. Adherent device 100 comprises a width 174, for example a maximum width along a width profile of adherent device 100. Width 174 can be from about 1 to about 4 inches (from about 25 mm to 100 mm), for example about 2 inches (about 50 mm).

FIGS. 1I and 1J show a side cross-sectional view and an exploded view, respectively, of adherent device 100 as in FIGS. 1A to 1H. Device 100 comprises several layers. Gel 114A, or gel layer, is positioned on electrode 112A to provide electrical conductivity between the electrode and the skin. Electrode 112A may comprise an electrode layer. Adhesive patch 110 may comprise a layer of breathable tape 110T, for example a known breathable tape, such as tricot-knit polyester fabric. An adhesive 116A, for example a layer of acrylate pressure sensitive adhesive, can be disposed on underside 110A of adherent patch 110. A gel cover 180, or gel cover layer, for example a polyurethane non-woven tape, can be positioned over patch 110 comprising the breathable tape. A PCB layer, for example flex printed circuit board 120, or flex PCB layer, can be positioned over gel cover 180 with electronic components 130 connected and/or mounted to flex printed circuit board 120, for example mounted on flex PCB so as to comprise an electronics layer disposed on the flex PCB layer. In many embodiments, the adherent device may comprise a segmented inner component, for example the PCB may be segmented to provide at least some flexibility. In many embodiments, the electronics layer may be encapsulated in electronics housing 160 which may comprise a waterproof material, for example silicone or epoxy. In many embodiments, the electrodes are connected to the PCB with a flex connection, for example trace 123A of flex printed circuit board 120, so as to provide strain relief between the electrodes 112A and 112D and the PCB. Gel cover 180 can inhibit flow of gel 114A and liquid. In many embodiments, gel cover 180 can inhibit gel 114A from seeping through breathable tape 110T to maintain gel integrity over time. Gel cover 180 can also keep external moisture, for example liquid water, from penetrating though the gel cover into gel 114A while allowing moisture vapor from the gel, for example moisture vapor from the skin, to transmit through the gel cover. In many embodiments, cover 162 can encase the flex PCB and/or electronics and can be adhered to at least one of the electronics, the flex PCB or adherent patch 110, so as to protect at least the electronics components and the PCB. Cover 162 can attach to adhesive patch 110 with adhesive 116B. Cover 162 can comprise many known biocompatible cover materials, for example silicone. Cover 162 can comprise an outer polymer cover to provide smooth contour without limiting flexibility. In many embodiments, cover 162 may comprise a breathable fabric. Cover 162 may comprise many known breathable fabrics, for example breathable fabrics as described above. In some embodiments, the breathable cover may comprise a breathable water resistant cover. In some embodiments, the breathable fabric may comprise polyester, nylon, polyamide, and/or elastane (Spandex) to allow the breathable fabric to stretch with body movement. In some embodiments, the breathable tape may contain and elute a pharmaceutical agent, such as an antibiotic, anti-inflammatory or antifungal agent, when the adherent device is placed on the patient.

The breathable cover 162 and adherent patch 110 comprises breathable tape can be configured to couple continuously for at least one week the at least one electrode to the skin so as to measure breathing of the patient. The breathable tape may comprise the stretchable breathable material with the adhesive and the breathable cover may comprises a stretchable material connected to the breathable tape, as described above, such that both the adherent patch and cover can stretch with the skin of the patient. Arrows 182 show stretching of adherent patch 110, and the stretching of adherent patch can be at least two dimensional along the surface of the skin of the patient. As noted above, connectors 122A and 122D between PCB 130 and electrodes 112A and 112D may comprise insulated wires that provide strain relief between the PCB and the electrodes, such that the electrodes can move with the adherent patch as the adherent patch comprising breathable tape stretches. Arrows 184 show stretching of cover 162, and the stretching of the cover can be at least two dimensional along the surface of the skin of the patient. For example, cover 162 and adhesive patch 110 can stretch in two dimensions along length 170 and width 174 with the skin of the patient, and stretching along length 170 can increase spacing between electrodes. Stretching of the cover and adhesive patch 110, for example in two dimensions, can extend the time the patch is adhered to the skin as the patch can move with the skin such that the patch remains adhered to the skin. Cover 162 can be attached to adherent patch 110 with adhesive 116B such that cover 162 stretches and/or retracts when adherent patch 110 stretches and/or retracts with the skin of the patient, for example along two dimensions comprising length 170 and width 174. Electronics housing 160 can be smooth and allow breathable cover 162 to slide over electronics housing 160, such that motion and/or stretching of cover 162 is slidably coupled with housing 160. The printed circuit board can be slidably coupled with adherent patch 110 that comprises breathable tape 110T, such that the breathable tape can stretch with the skin of the patient when the breathable tape is adhered to the skin of the patient. Electronics components 130 can be affixed to printed circuit board 120, for example with solder, and the electronics housing can be affixed over the PCB and electronics components, for example with dip coating, such that electronics components 130, printed circuit board 120 and electronics housing 160 are coupled together. Electronics components 130, printed circuit board 120, and electronics housing 160 are disposed between the stretchable breathable material of adherent patch 110 and the stretchable water resistant material of cover 160 so as to allow the adherent patch 110 and cover 160 to stretch together while electronics components 130, printed circuit board 120, and electronics housing 160 do not stretch substantially, if at all. This decoupling of electronics housing 160, printed circuit board 120 and electronic components 130 can allow the adherent patch 110 comprising breathable tape to move with the skin of the patient, such that the adherent patch can remain adhered to the skin for an extended time of at least one week, for example two or more weeks.

An air gap 169 may extend from adherent patch 110 to the electronics module and/or PCB, so as to provide patient comfort. Air gap 169 allows adherent patch 110 and breathable tape 110T to remain supple and move, for example bend, with the skin of the patient with minimal flexing and/or bending of printed circuit board 120 and electronic components 130, as indicated by arrows 186. Printed circuit board 120 and electronics components 130 that are separated from the breathable tape 110T with air gap 169 can allow the skin to release moisture as water vapor through the breathable tape, gel cover, and breathable cover. This release of moisture from the skin through the air gap can minimize, and even avoid, excess moisture, for example when the patient sweats and/or showers.

The breathable tape of adhesive patch 110 may comprise a first mesh with a first porosity and gel cover 180 may comprise a breathable tape with a second porosity, in which the second porosity is less than the first porosity to minimize, and even inhibit, flow of the gel through the breathable tape. The gel cover may comprise a polyurethane film with the second porosity.

In many embodiments, the adherent device comprises a patch component and at least one electronics module. The patch component may comprise adhesive patch 110 comprising the breathable tape with adhesive coating 116A, at least one electrode, for example electrode 114A and gel 114. The at least one electronics module can be separable from the patch component. In many embodiments, the at least one electronics module comprises the flex printed circuit board 120, electronic components 130, electronics housing 160 and cover 162, such that the flex printed circuit board, electronic components, electronics housing and cover are reusable and/or removable for recharging and data transfer, for example as described above. In many embodiments, adhesive 116B is coated on upper side 110A of adhesive patch 110B, such that the electronics module can be adhered to and/or separated from the adhesive component. In specific embodiments, the electronic module can be adhered to the patch component with a releasable connection, for example with Velcro™, a known hook and loop connection, and/or snap directly to the electrodes. Two electronics modules can be provided, such that one electronics module can be worn by the patient while the other is charged, as described above. For example, about 12 patches can be used to monitor the patient for at least 90 days with at least one electronics module, for example with two reusable electronics modules.

At least one electrode 112A can extend through at least one aperture 180A in the breathable tape 110 and gel cover 180.

In some embodiments, the adhesive patch may comprise a medicated patch that releases a medicament, such as antibiotic, beta-blocker, ACE inhibitor, diuretic, or steroid to reduce skin irritation. The adhesive patch may comprise a thin, flexible, breathable patch with a polymer grid for stiffening. This grid may be anisotropic, may use electronic components to act as a stiffener, may use electronics-enhanced adhesive elution, and may use an alternating elution of adhesive and steroid.

FIG. 1K shows at least one electrode 190 configured to electrically couple to a skin of the patient through a breathable tape 192. In many embodiments, at least one electrode 190 and breathable tape 192 comprise electrodes and materials similar to those described above. Electrode 190 and breathable tape 192 can be incorporated into adherent devices as described above, so as to provide electrical coupling between the skin and electrode through the breathable tape, for example with the gel.

Second adherent device 100J and third adherent device 100A may comprise components similar to adherent device 100, described above. The processor of adherent device 100, described above may comprise a system controller to control communication and/or actions of first adherent device 100J and second device 100A, for example data collection and transmission. In many embodiments, data collected from second adherent device 100J and third adherent device 100A is sent wirelessly to device 100, which device 100 transmits the data to the intermediate device.

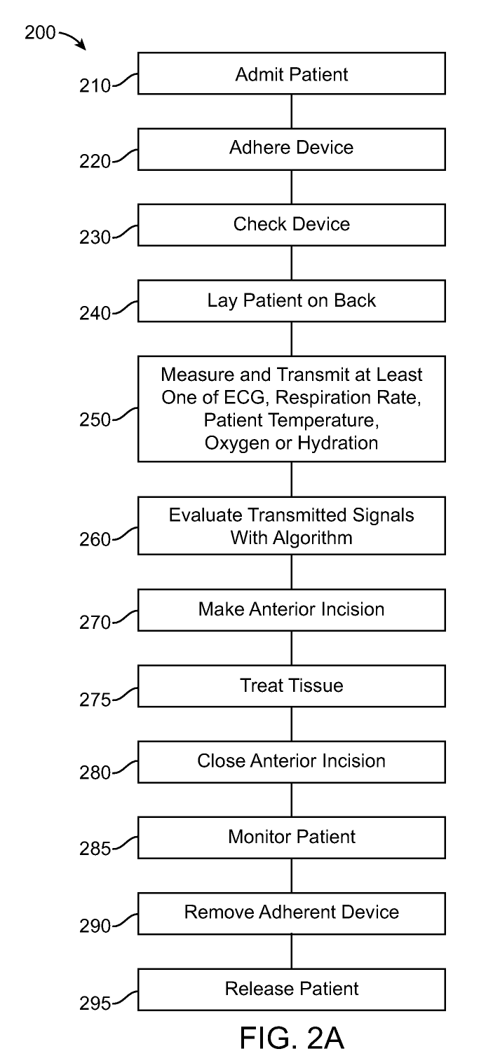

FIG. 2A shows a method of monitoring and/or treating a patient, according to embodiments of the present invention. A step 210 admits a patient, for example a patient admitted to the hospital by hospital personnel. The patient can be registered and the device associated with the patient when the patient is registered, for example associated by unique device ID number. A step 220 adheres the device to the back of the patient, for example on the lower back or between the shoulder blades as described above. A step 240 checks the device, for example by collecting patient data to verify that the device is working properly. A step 240 lays the patient on his or her back in the supine position with the device placed on and adhered to the back of the patient. A step to 250 measures and transmits at least one of an ECG signal, an impedance measurement signal, a respiration rate signal, a patient temperature signal, a patient oxygen signal or a hydration signal. A step 260 evaluates the transmitted signals with an algorithm, for example an arrhythmia detection algorithm. A step 270 makes an anterior incision in the patient, for example through the skin of the chest, when the patient lies in the supine position. A step 270 treats tissue, for example treats heart tissue with bypass surgery. A step 280 closes the anterior incision. A step 285 monitors the patient, for example when the patient recovers. A step 290 removes the adherent device from the back of the patient, for example after the patient has recovered. A step 295 releases the patient.

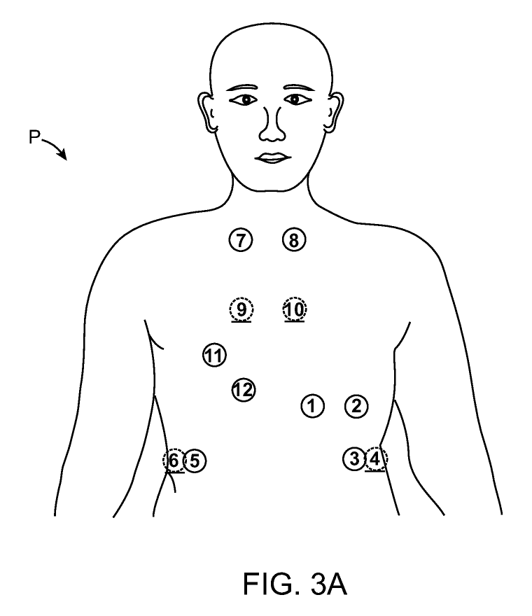

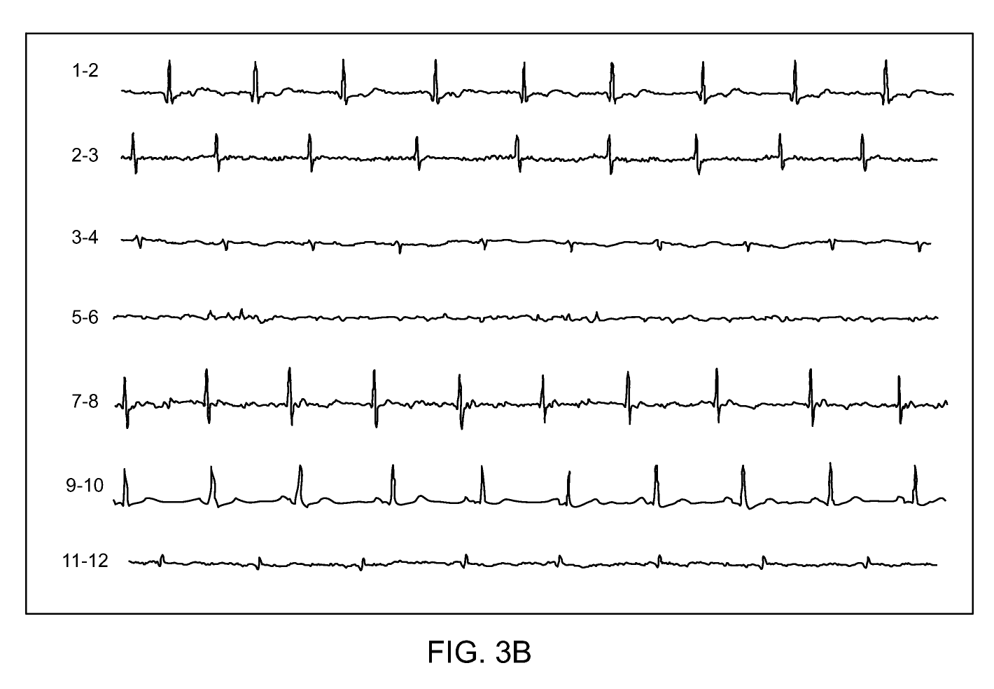

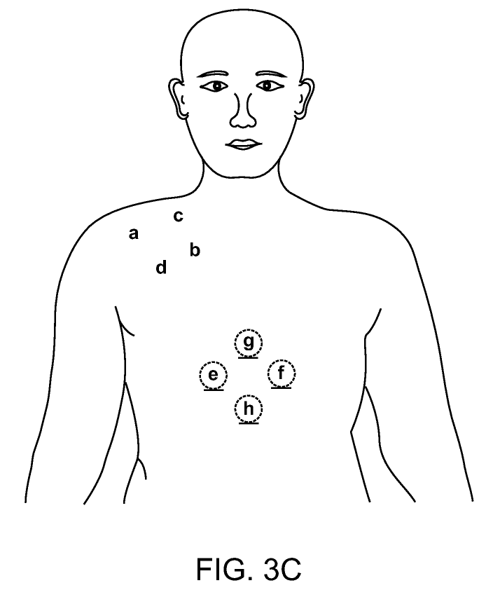

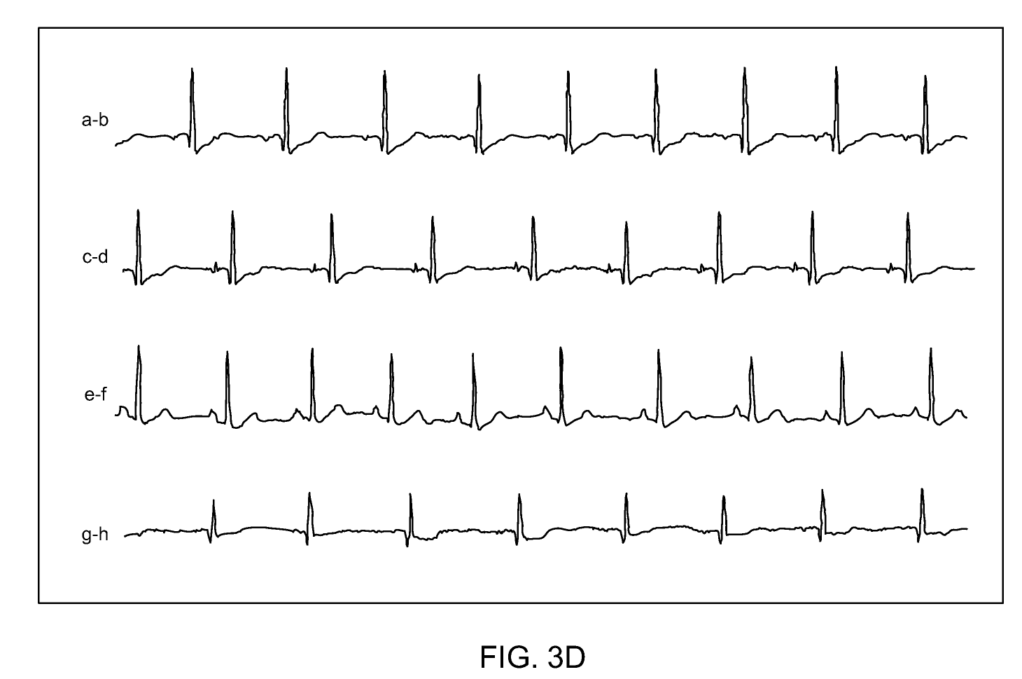

It should be appreciated that the specific steps illustrated in FIG. 2A provide a particular method of monitoring and/or treating a patient, according to an embodiment of the present invention. Other sequences of steps may also be performed according to alternative embodiments. For example, alternative embodiments of the present invention may perform the steps outlined above in a different order. Moreover, the individual steps illustrated in FIG. 3 may include multiple sub-steps that may be performed in various sequences as appropriate to the individual step. Furthermore, additional steps may be added or removed depending on the particular applications. One of ordinary skill in the art would recognize many variations, modifications, and alternatives.

Experimental Results

FIG. 3A shows electrode positioning for experiments measuring ECG signals on patient P. Electrode positions 1, 2, 3, 5, 7, 8, 11 and 12 are located on the anterior side, or front, of the patient. Electrode positions 4, 6, 9 and 10 are located on the posterior side, or back, of the patient.

FIG. 3B show ECG signals with electrode positions as in FIG. 3A. Signal 1-2 taken with electrodes at positions 1 and 2 shows a good quality ECG control signal measured from the front of the patient. Signal 2-3 taken with electrodes at positions 2 and 3 shows a good quality ECG control signal measured from the front of the patient. Signal 3-4 taken with electrodes at positions 3 and 4 on the front and back sides of the patient show ECG signals with less desirable characteristics. Signal 5-6 taken with electrodes at positions 5 and 6 on the front and back sides of the patient show ECG signals with less desirable characteristics. Signal 7-8 taken with electrodes at positions 7 and 8 a good quality ECG control signal measured from the front of the patient. Signal 9-10 taken with electrodes at positions 9 and 10 shows a good quality ECG signal from the back of the patient and shows that placement of ECG electrodes on the back of the patient can produce a quality ECG signal. Signal 11-12 taken with electrodes at positions 11 and 12 shows an ECG signal with less desirable characteristics. The above measurements are merely examples. Similar measurements can be obtained for additional ECG signals and/or impedance signals with empirical measurements on a patient population, for example of 10 patients.

FIG. 3C shows electrode positioning for experiments measuring ECG signals, according to embodiments of the present invention. Electrode positions a, b, c and d are located on the anterior side, or front, of the patient. Electrode positions e, f, g and h are located on the posterior side, or back, of the patient. One will appreciate that positions e and f are symmetrically disposed about the midline of the patient with a substantially similar distance from the midline to each of position e and position f, respectively. One will also appreciate that position g and position h are each disposed along the midline of the patient.

FIG. 3D show ECG signals with electrode positions as in FIG. 3C. Signal a-b taken with electrodes at positions a and b shows a good quality ECG control signal measured from the front of the patient. Signal c-d taken with electrodes at positions c and d shows a good quality ECG control signal measured from the front of the patient. Signal e-f taken with electrodes at positions e and f shows a good quality ECG signal from the back of the patient and shows that placement of ECG electrodes on the back of the patient can produce a quality ECG signal. Signal g-h taken with electrodes at positions g and h along the midline of the patient shows a good quality ECG signal from the back of the patient and shows that placement of ECG electrodes on the back of the patient can produce a quality ECG signal. However, the g-h signal has a slightly smaller amplitude than the e-f signal and the known QRS waveform is somewhat less well defined than e-f signal, so as to indicate that optimal ECG measurement results can be obtained with electrodes disposed on opposite sides of the midline, for example as with the e-f signal. The above measurements are merely examples. Similar measurements can be obtained for additional ECG signals and/or impedance signals with empirical measurements on a patient population, for example of 10 patients.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. A method of monitoring a patient having a back and a spine disposed along a midline of the patient, the method comprising:
placing a first electrode and a second electrode on the back of the patient; and
measuring a signal from the first electrode and the second electrode.

2. The method of claim 1 wherein the first electrode and the second electrode are placed such that the first electrode is placed on a first side of the midline and the second electrode is placed on a second side of the midline opposite the first side

3. The method of claim 1 wherein the first electrode and the second electrode are placed along the midline and aligned with the spine.

4. The method of claim 1 wherein the first electrode and the second electrode are placed on at least one of a lower back or between shoulder blades of the patient.

5. The method of claim 4 wherein the first electrode is placed a first distance from the midline of the patient and the second electrode is placed a second distance from the midline of the patient, the first distance substantially similar to the second distance such that the first electrode and the second electrode are symmetrically disposed on opposite sides of the midline of the patient.

6. The method of claim 1 wherein the signal comprises at least one of an electrocardiogram signal or a bioimpedance signal.

7. The method of claim 6 wherein the signal comprises the bioimpedance signal and at least one of a hydration or a respiration of the patient is determined from the bioimpedance signal.

8. The method of claim 1 wherein the first electrode and the second electrode are supported with a flexible adherent device comprising a support adhered to the back of the patient.

9. The method of claim 8 wherein the support comprises a midline and the midline of the support is aligned with the midline of the patient when the support is adhered to the back of the patient.

10. The method of claim 9 wherein the support supports rigid circuitry components disposed on each side of the support away from the midline of the support to minimize pressure to the spine of the patient when the patient is placed in the supine position.

11. The method of claim 10 wherein the rigid circuitry components comprise at least one of an integrated circuit or a rigid printed circuitry board.

12. The method of claim 8 wherein the flexible adherent device is adhered to at least one of a lower back of the patient or an upper back of the patient.

13. The method of claim 12 wherein the device is adhered to the lower back of the patient and at least partially supports a lumbar of the spine of the patient when the patient sits and/or is placed in a supine position.

14. The method of claim 12 wherein the support is adhered to the back between shoulder blades of the patient.

15. The method of claim 1 wherein the flexible adherent device comprises a central portion supported on the back with the skin of the patient, the central portion comprising a midline aligned with the midline of the patient.

16. The method of claim 15 wherein the flexible adherent device comprises peripheral portions adhered to the back and extending from the central portion.

17. The method of claim 16 wherein the central portion comprises a thickness of no more than about 10 mm from an inner surface configured to adhere to the skin to an outer surface opposite the inner surface and wherein the peripheral portions each comprise a thickness of no more than about 5 mm from the inner surface configured to adhere to the skin of the patient to the outer surface opposite the inner surface.

18. The method of claim 17 wherein the thickness of the central portion comprises no more than about 5 mm and the thickness of each peripheral portion comprises no more than about 3 mm.

19. The method of claim 15 wherein the central portion comprises the midline aligned with the spine and wherein the peripheral portions comprise the electrodes and extend along at least one of trapezius muscles or latissimus dorsi muscles of the patient.

20. The method of claim 1 wherein a second signal is measured and comprises at least one of an activity signal, a posture signal, a temperature signal or an oxygen saturation signal.

21. The method of claim 1 further comprising transmitting the signal wirelessly to a monitoring station in a hospital such that hospital personnel can monitor a status of the patient.

22. The method of claim 1 wherein an algorithm is configured to determine a condition of the patient in response to the signal and generate an alarm in response to the condition.

23. The method of claim 22 wherein the condition comprises an arrhythmia of the patient.

24. A device for monitoring a patient having a back, the device comprising:
a support comprising an adhesive, the support configured to adhere to a skin of the back of the patient;
at least two electrodes supported with the support; and
circuitry supported with the support and coupled to the at least two electrodes, the circuitry configured to measure a signal from the electrodes and transmit the signal wirelessly.

25. The device of claim 24 wherein support is configured to flex and conform to the surface contour of the skin of the patient when the support is adhered to the skin and wherein the support comprises at least one of a breathable tape or a flex printed circuit board configured to flex with the surface contour of the skin of the patient when the support is adhered to the skin of the patient.

26. The device of claim 24 wherein support comprises a midline configured for alignment with a spine of the patient.

27. The device of claim 26 wherein support comprises a visible indicia to align the support with the midline of the patient along the spine.

28. The device of claim 24 wherein a first portion of the circuitry is disposed on first side of the midline and a second portion of the circuitry is disposed on a second side of the midline opposite the first side.

29. The device of claim 24 wherein the device comprises a thickness profile comprising a distance extending from a lower surface of the device configured for placement against the skin of the patient to an outer surface of the device opposite the lower surface and wherein the thickness profile comprises a first central dimension at a first location configured for placement on the midline of the patient and a second peripheral dimension at a second location configured for placement away from the midline of the patient, the first central dimension greater than the second peripheral dimension.

30. The device of claim 29 wherein the thickness profile comprises a third distance at a third location, the third location located away from the midline and between the first location and the second location, the third distance greater than the first distance and the second distance.

31. The device of claim 24 further comprising:
a cover having an outer surface, the cover disposed over the electronics and supported with the support and wherein the cover and support comprise a central portion of the device and peripheral portions of the device, each portion having a thickness extending from the adhesive to the outer surface of the cover, the central portion comprising the midline configured for alignment with the spine of the patient and having a maximum thickness no more than about 10 mm, the peripheral portions extending from the central portion and having a maximum thickness of no more than about 5 mm.

32. The device of claim 31 wherein the cover comprises at least one of a coating, a dip coating, a molding, a housing, a casing or a stretchable fabric.

33. The device of claim 31 wherein the maximum thickness of the central portion comprises no more than about 5 mm and the maximum thickness of each peripheral portion comprises no more than about 3 mm.

34. The device of claim 31 wherein the maximum thickness of the central portion is disposed away from a midline of the central portion and wherein the central portion comprises a second thickness along the midline less than the maximum thickness of the central portion to decrease pressure on a spine of the patient when the patient lies in a supine position.

35. The device of claim 24 wherein the support comprises a midline and extends away from the midline symmetrically to an outer boundary disposed symmetrically about the midline and wherein the at least two electrodes are positioned on the support symmetrically about the midline such that the electrodes are positioned on opposite sides of the spine at equal distances from the midline when the supported is adhered to the back of the patient and the midline of the support is aligned with the spine of the patient.

36. The device of claim 24 wherein support comprises at least one of a breathable tape or a flex printed circuit board configured to stretch with the skin of the patient.

37. The device of claim 24 wherein the support is shaped for lumbar support of the patient.

38. A system for monitoring a patient having a back, the system comprising:
at least one support comprising an adhesive, the at least one support configured to adhere to a skin of the back of the patient;
at least two electrodes supported with the at least one support;
circuitry supported with the at least one support and coupled to the at least two electrodes, the circuitry configured to measure a signal from the electrodes and transmit a wireless signal comprising the signal from the electrodes; and
a gateway configured to receive the wireless signal.

39. The system of claim 38 wherein the circuitry is configured to monitor and transmit wirelessly to the gateway at least one of an electrocardiogram signal, a respiration rate signal, body fluid signal, an activity signal, a posture signal, a temperature signal or an oxygen saturation signal.

40. The system of claim 38 further comprising:
at least one processor comprising a tangible medium configured to receive the signal from the gateway; and
a display located at a station to monitor the patient and coupled to the at least one processor to display the signal.

41. The system of claim 38 wherein the at least one support comprises a first support and a second support and the circuitry comprises first circuitry supported with the first support and second circuitry supported with the second support, the first circuitry configured to measure a first signal from the patient, the second circuitry configured to measure a second signal from the patient, the first circuitry and the second circuitry each configured to transmit signals to the gateway, the first support configured to adhere to at least one of a lower back of the patient or between shoulder blades of the patient.

42. The system of claim 41 wherein the first circuitry is coupled to a first at least two electrodes configured to measure the first signal comprising a first cardiac vector in a direction extending between the first at least two electrodes, the second circuitry coupled to a second at least two electrodes configured to measure the second signal comprising a second cardiac vector in a direction extending between the second at least two electrodes.

43. The system of claim 42 wherein the first at least two electrodes extend substantially laterally across the back of the patient and the second at least two electrodes extend substantially vertically along at least one of the back or the side of the patient.
